(19) United States
(12) Patent Application Publication
(10) Pub. No.: US 2018/0333173 A1
(43) Pub. Date: Nov. 22, 2018

(54) SYSTEMS AND METHODS FOR MAKING AND USING A LEAD INTRODUCER FOR AN ELECTRICAL STIMULATION SYSTEM

(71) Applicant: Boston Scientific Neuromodulation Corporation, Valencia, CA (US)

(72) Inventor: Kevin Peng Wang, Fremont, CA (US)

(21) Appl. No.: 15/980,297

(22) Filed: May 15, 2018

Related U.S. Application Data
(60) Provisional application No. 62/509,574, filed on May 22, 2017.

(51) Int. Cl. A61B 17/34; A61N 1/05

(52) U.S. Cl. CPC A61B 17/3468; A61B 17/3417; A61B 2017/00526; A61N 1/0551; A61B 17/3474

(57) ABSTRACT

A lead introducer includes a needle assembly and a hub assembly. The needle assembly includes an outer needle defining an outer-needle open channel; and an inner needle disposed within the outer needle and defining an inner-needle open channel. The inner needle is rotatable independently within the outer needle to transition the needle assembly between open and closed positions. When in the open position, the inner-needle open channel is rotationally-aligned with the outer-needle open channel to form a separation channel for separating a lead from the needle assembly. When in the closed position, the inner-needle open channel is rotationally-offset from the outer-needle open channel to form a lead lumen. The hub assembly includes an outer-needle hub coupled to the outer needle and an inner-needle hub coupled to the inner needle.

### CROSS-REFERENCE TO RELATED APPLICATIONS

This application claims the benefit under 35 U.S.C. § 119(e) of U.S. Provisional Patent Application Ser. No. 62/509,574, filed May 22, 2017, which is incorporated herein by reference.

### FIELD

The present invention is directed to the area of implantable electrical stimulation systems and methods of making and using the systems. The present invention is also directed to a lead introducer for facilitating insertion of implantable electrical stimulation leads into patients, as well as methods of making and using the lead introducers and electrical stimulation leads.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a lead introducer that includes a needle assembly and a hub assembly. The needle assembly has a distal portion, a proximal portion, and a longitudinal length. The needle assembly includes an outer needle and an inner needle. The outer needle defines an outer-needle open channel extending longitudinally along the needle assembly. The inner needle defines an inner-needle open channel extending longitudinally along the needle assembly. The inner needle is disposed within the outer needle and rotatable independently within the outer needle to transition the needle assembly between an open position and a closed position. When the needle assembly is in the open position, the inner-needle open channel is rotationally-aligned with the outer-needle open channel to form a separation channel for separating a lead disposed in the needle assembly from the needle assembly. When the needle assembly is in the closed position, the inner-needle open channel is rotationally-offset from the outer-needle open channel and the outer needle and inner needle define a lead lumen within the needle assembly. The hub assembly is coupled to the proximal portion of the needle assembly. The hub assembly includes an outer-needle hub coupled to the outer needle and an inner-needle hub coupled to the inner needle.

In at least some embodiments, an inflation needle is configured and arranged to slide within the inner needle. In at least some embodiments, a stylet is configured and arranged to slide within the inflation needle. In at least some embodiments, the distal portion of the needle assembly includes a sharpened cutting surface configured and arranged to facilitate piercing of patient tissue. In at least some embodiments, a sheath is configured and arranged for disposing over the needle assembly.

In at least some embodiments, a blade extends outwardly from the needle assembly, the blade configured and arranged to slide longitudinally along the needle assembly and cut away patient tissue in proximity to the needle assembly. In at least some embodiments, the blade is attached to the inflation needle. In at least some embodiments, the blade is attached to a cutting member configured and arranged to slide within the inner needle.

In at least some embodiments, the hub assembly includes a rotation actuator for facilitating manual transitioning of the needle assembly between the open position and the closed position by a user of the lead introducer. In at least some embodiments, the hub assembly includes a rotation-control feature coupled to the rotation actuator, the rotation-control feature configured and arranged for facilitating transitioning of the needle assembly between the open position and the closed position. In at least some embodiments, the rotation-control feature is configured and arranged to releasably lock the needle assembly in at least one of the open position or the closed position. In at least some embodiments, the rotation-control feature includes a biasing member configured and arranged for biasing transitioning of the needle assembly between the open position and the closed position.

In at least some embodiments, the lead introducer is configured and arranged for passing a lead disposed in the needle assembly through the separation channel by displacing the lead with the inflation needle.

Another embodiment is an insertion kit that includes the lead introducer described above and an electrical stimulation lead having electrodes disposed along a distal portion of the electrical stimulation lead.

Yet another embodiment is an electrical stimulation system that includes the insertion kit described above and a control module coupleable to the electrical stimulation lead. The control module includes a housing and an electronic subassembly disposed in the housing.

Still yet another embodiment is a method for implanting an electrical stimulation lead into a patient. The method includes (a) advancing the lead introducer described above into the patient; (b) removing the inflation needle from the patient, leaving the inner needle and outer needle within the patient, the inner needle and outer needle being in the closed position, thereby forming the lead lumen extending along the longitudinal length of the needle body; (c) inserting into the lead lumen a first electrical stimulation lead including electrodes disposed along a distal portion of the first electrical stimulation lead and terminals disposed along a proximal portion of the first electrical stimulation lead; (d) rotating the inner needle relative to the outer needle to transition the needle assembly to the open position, thereby forming the separation channel extending along the longitudinal length of the needle body; and (e) removing the needle assembly from the patient, leaving the first electrical stimulation lead within the patient.

In at least some embodiments, the method further includes cutting away patient tissue in proximity to the needle assembly using a blade extending outwardly from within the needle assembly.

In at least some embodiments, (d) rotating the inner needle relative to the outer needle to transition the needle assembly to the open position includes using a rotation actuator.

In at least some embodiments, the method further includes reinserting, subsequent to performing step (d) and prior to performing step (e), the inflation needle into the lead lumen, the reinsertion of the inflation needle into the lead lumen displacing the first electrical stimulation lead from the needle assembly, thereby causing the first electrical stimulation lead to separate from the needle assembly through the separation channel.

In at least some embodiments, the method further includes transitioning, subsequent to performing step (d) and prior to performing step (e), the needle assembly to the closed position; removing the inflation needle from the needle assembly; inserting into the lead lumen a second electrical stimulation lead including electrodes disposed along a distal portion of the second electrical stimulation lead and terminals disposed along a proximal portion of the second electrical stimulation lead; and transitioning the needle assembly to the open position.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic perspective view of one embodiment of a lead introducer suitable placing the lead of FIG. 1 into a patient, according to the invention;

FIG. 2B is a schematic transverse cross-sectional view of one embodiment of a needle assembly of the lead introducer of FIG. 2A, according to the invention;

FIGS. 3A-3H are schematic transverse cross-sectional views of one embodiment of the needle assembly of FIG. 2B undergoing an implantation procedure to implant a first lead, according to the invention;

FIGS. 4A-4E are schematic transverse cross-sectional views of one embodiment of the needle assembly of FIG. 2B undergoing an implantation procedure to implant a second lead, the needle assembly remaining in the same location for the implantation procedure of FIGS. 3A-3H and the implantation procedure of FIGS. 4A-4E, according to the invention;

FIG. 5 is a schematic transverse cross-sectional view of one embodiment of a sheath disposed around the needle assembly of FIG. 2B, according to the invention;

FIG. 6 is a schematic transverse cross-sectional view of one embodiment of a blade extending outwardly from a side of the needle assembly of FIG. 2A, according to the invention;

FIG. 7 is a schematic perspective view of one embodiment of the blade of FIG. 6 disposed along a distal portion of an inflation needle of the needle assembly of FIG. 6; according to the invention; and

FIG. 8 is a schematic perspective view of one embodiment of the distal end of the inflation needle of FIG. 7 nested in an inner needle of the needle assembly of FIG. 6, according to the invention.

### DETAILED DESCRIPTION

The present invention is directed to the area of implantable electrical stimulation systems and methods of making and using the systems. The present invention is also directed to a lead introducer for facilitating insertion of implantable electrical stimulation leads into patients, as well as methods of making and using the lead introducers and electrical stimulation leads.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, cuff leads, or any other arrangement of electrodes on a lead. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference. In the discussion below, a percutaneous lead will be exemplified, but it will be understood that the methods and systems described herein are also applicable to paddle leads and other leads.

A percutaneous lead for electrical stimulation (for example, deep brain, spinal cord, peripheral nerve, or cardiac-tissue stimulation) includes stimulation electrodes that can be ring electrodes, segmented electrodes that extend only partially around the circumference of the lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues.

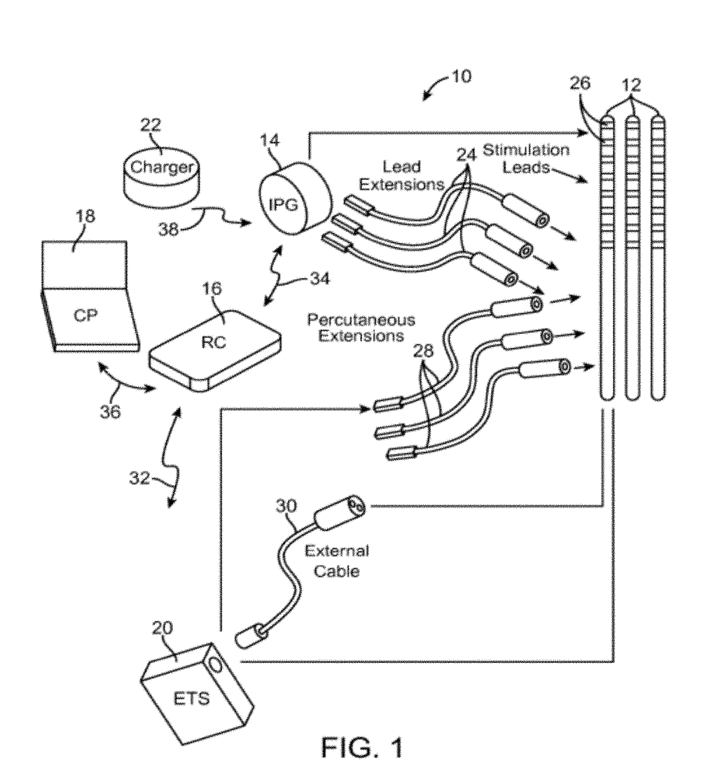

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity. The implantable pulse generator can have eight stimulation channels which may be independently programmable to control the magnitude of the current stimulus from each channel. In some embodiments, the implantable pulse generator can have more or fewer than eight stimulation channels (e.g., 4-, 6-, 16-, 32-, or more stimulation channels). The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programmed via wireless communications (e.g., Bluetooth) between the RC 16 (or external device such as a hand-held electronic device) and the IPG 14.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and U.S. Pat. Nos. 7,761,165; 7,974,706; 8,175,710; 8,224,450; and 8,364,278; and U.S. Patent Application Publication No. 2007/0150036, as well as the other references cited above, all of which are incorporated by reference.

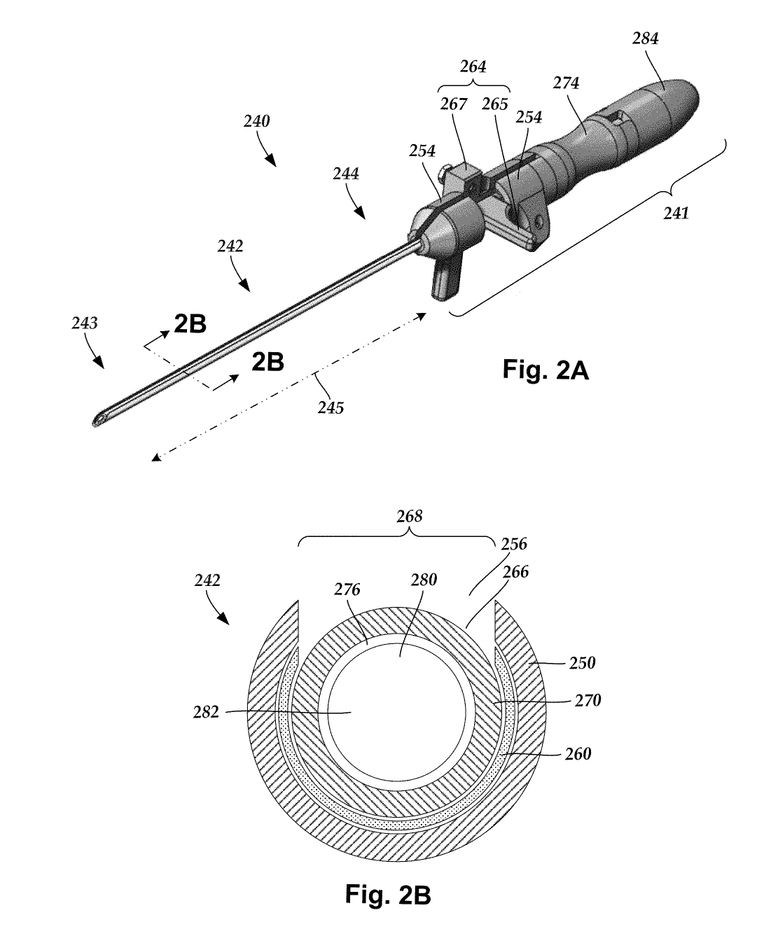

Turning to FIGS. 2A-2B, some percutaneous-lead implantation procedures involve inserting a lead introducer, such as an epidural needle, into a patient. Once the lead introducer is inserted into the patient and advanced to, or in proximity to, a target stimulation location, a lead is inserted into the lead introducer and advanced along the lead introducer. The lead introducer is then removed from the patient, leaving the lead in place. Typically, the lead introducer is removed from the patient by sliding the lead introducer off the proximal end of the lead.

In the case of spinal cord stimulation, the lead introducer is typically used for introducing the lead into the epidural space. In some instances, multiple leads may be implanted into a patient. For example, a patient may receive treatment, via spinal cord stimulation, where the stimulated spinal cord levels are spaced apart from one another such that electrodes of a single lead cannot safely, and concurrently, stimulate the patient at each of the spinal cord levels. When multiple leads are being placed, multiple lead introducers, or multiple needle pokes from the same lead introducer, may be needed, as conventional lead introducers are limited to inserting a single lead at a time.

As described herein, a lead introducer is designed to place multiple leads from a single lead-introducer with a single insertion into a patient without needing to be removed from the patient and reinserted between consecutive lead placements. It is an advantage to reduce the number of lead-introducer insertions into the patient. Placing leads into a patient carries a risk of the patient developing an infection. Each needle insertion exposes the patient to a risk of infection as the needle is exposed to contamination when external to the patient and not contained in a sterile environment.

Placing multiple leads into a patient with a single needle insertion may also reduce surgery time. Surgery time may be reduced because medical practitioners do not need to take the time to remove and reinsert the insertion needle between lead placements. Additionally, surgery time may be reduced by obviating the need to tunnel between multiple lead-introducer-insertion locations to bridge the distance between the lead-introducer-insertion locations. Furthermore, surgery time may be reduced by obviating the need for the patient's epidural space to be found during each lead-introducer insertion.

FIG. 2A shows, in perspective view, one embodiment of a lead introducer 240 configured and arranged to facilitate implantation of one or more electrical stimulation leads into a patient. The lead introducer 240 includes a needle assembly 242 coupled to a hub assembly 241. The needle assembly 242 has a distal portion 243, a proximal portion 244, and a longitudinal length 245. The hub assembly 241 includes an outer-needle hub 254, an inner-needle hub 264, and an inflation-needle hub 274.

FIG. 2B illustrates, in transverse cross-sectional view, one embodiment of the needle assembly 242. The needle assembly 242 includes an outer needle 250, an inner needle 260, and an inflation needle 270. The needles 250, 260, 270 are nest-able. Each of the hubs, 254, 264, 274 is coupled to a different one of the needles 250, 260, and 270, respectively, along the proximal portion 244 of the needle assembly 242.

The outer needle 250 defines an outer-needle open channel 256 extending along the longitudinal length 245 of the needle assembly 242. In at least some embodiments, the outer-needle open channel 256 also extends along at least a portion of the outer-needle hub 254.

The inner needle 260 is disposed within the outer needle 250. The inner needle 260 is rotatable relative to the outer needle 250 about the longitudinal length 245 of the needle assembly 242 when the inner needle 260 is at least partially nested within the outer needle 250. In at least some embodiments, the inner needle 260 is configured to slide along the longitudinal length 245 of the needle assembly 242 independently from the outer needle 250. The inner needle 260 defines an inner-needle open channel 266 extending along the longitudinal length 245 of the needle assembly 242. In at least some embodiments, the inner-needle open channel 266 also extends along at least a portion of the inner-needle hub 264.

Rotation of the inner needle 260 relative to the outer needle 250 causes the needle assembly 242 to transition between an open position and a closed position. When the needle assembly 242 is in the open position the inner-needle open channel 266 is rotationally-aligned with the outer-needle open channel 256 to form a separation channel 268 extending along the longitudinal length 245 of the needle assembly 242 and, optionally, along at least a portion of one or more of the outer-needle hub 254 or the inner-needle hub 264. The separation channel 268 facilitates placement of a lead at, or in proximity to, a target stimulation location within a patient by enabling the lead to pass through the separation channel 268 from inside the needle assembly to a location external to the needle assembly, thereby separating from the lead introducer.

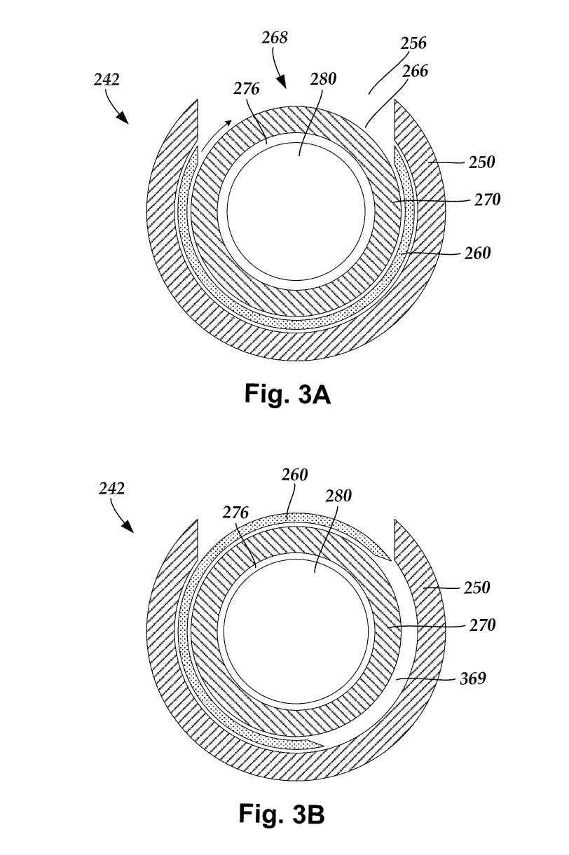

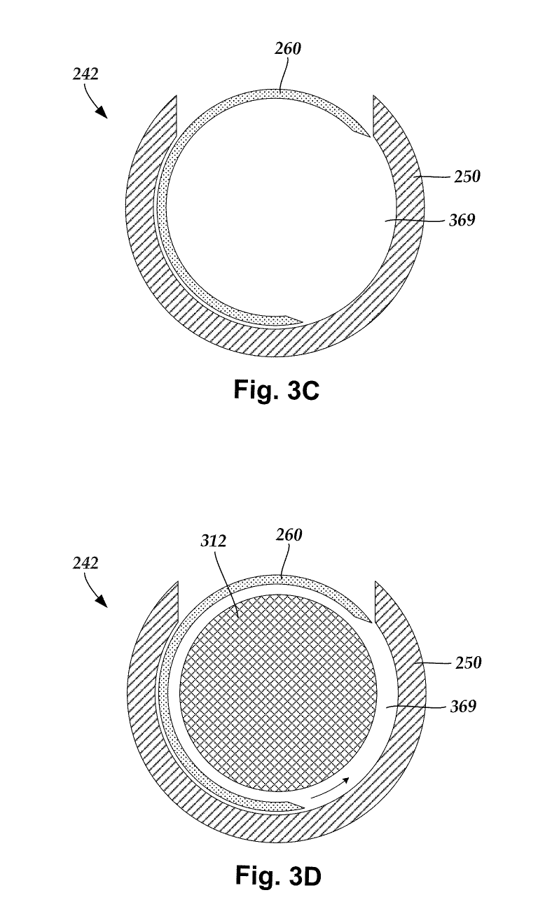

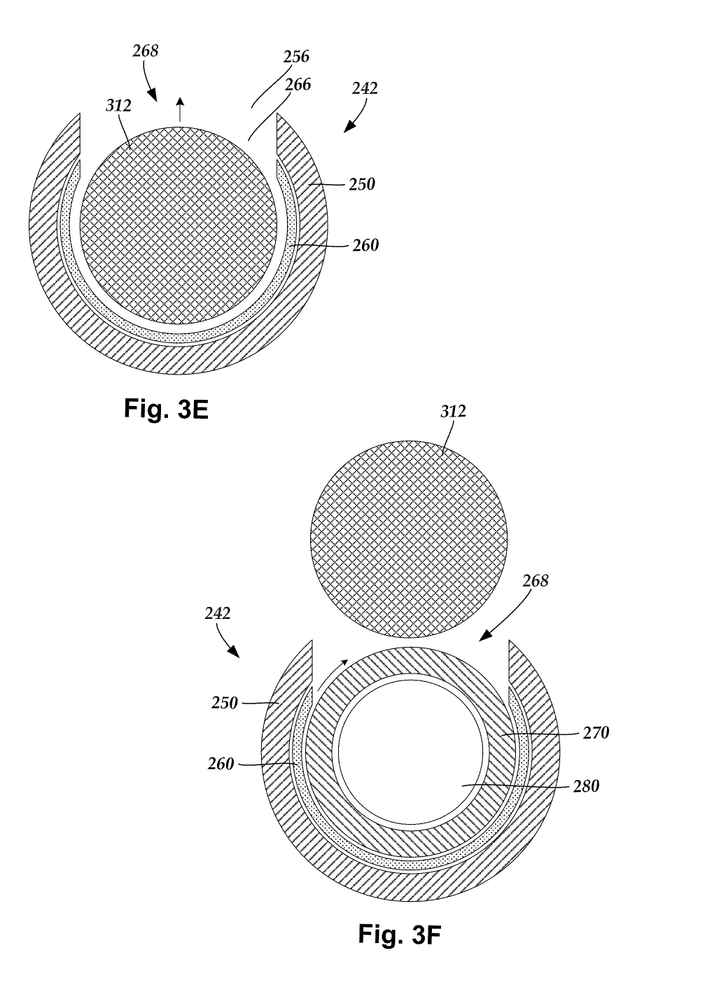

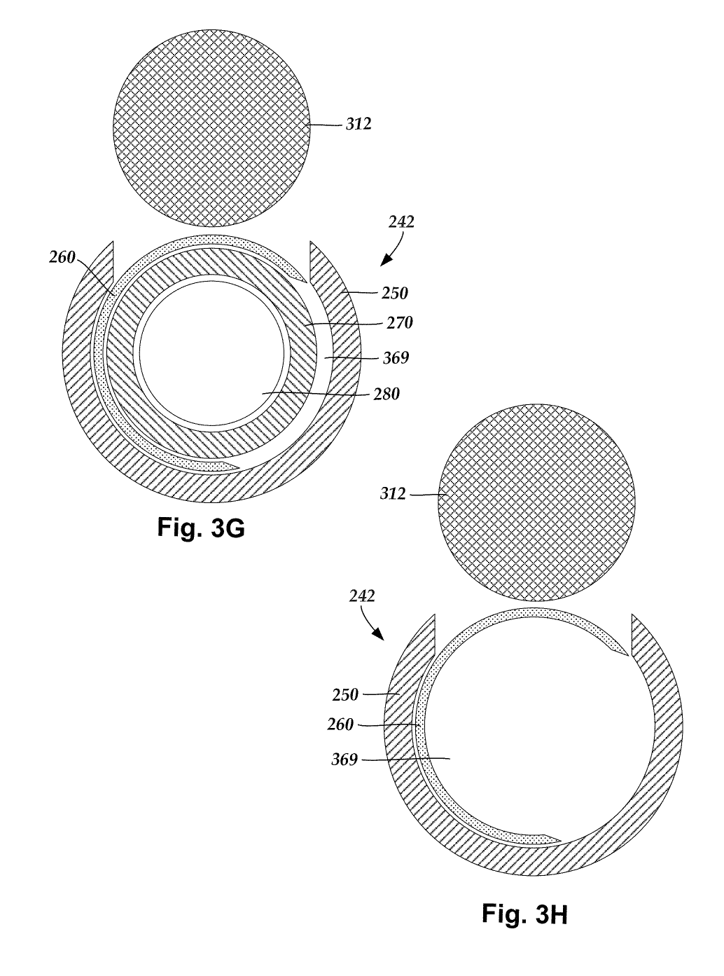

When the needle assembly 242 is in the closed position, the inner-needle open channel 266 is rotationally-offset from the outer-needle open channel 256 along the longitudinal length 245 of the needle assembly 242, thereby forming a lead lumen (see e.g., 369 in FIG. 3D) extending along the longitudinal length 245 of the needle assembly 242. The lead lumen is suitable for retaining an inserted lead.

In the illustrated embodiment, the inner-needle hub 264 includes an optional rotation actuator 265 (e.g., a lever or trigger) that can be manually used by a user of the lead introducer for facilitating rotation of the inner needle 260 relative to the outer needle 250. In the illustrated embodiment, the inner-needle hub 264 includes an optional rotation-control feature 267. In at least some embodiments, the rotation-control feature 267 is attached to the hub assembly 241 and coupled with the rotation actuator 265 to facilitate control of rotation of the inner needle relative 260 to the outer needle 250.

In at least some embodiments, the rotation-control feature 267 is configured to lock the needle assembly 242 in an open position, a closed position, or both. The rotation-control feature 267 can be biased to control the amount of rotation of the inner needle relative to the outer needle, or the amount of resistance to rotation. In at least some embodiments, the rotation-control feature 267 includes one or more biasing members (e.g., extension/compression springs, torsional springs, or the like).

Optionally, it may be desirable to automate, or partially automate, one or more steps of the above-described implantation technique for implanting one or more leads. The inclusion of an automated system can be implemented using one or more biasing members and one or more actuators. For example, potential energy can be stored in one or more biasing members that is activated by pulling a trigger (or pushing a button, pulling a switch, lever, or the like). The automated system can, optionally, be pneumatic and use compressed air cartridges in addition to, or in lieu of, using one or more biasing members.

The inflation needle 270 is disposed within the inner needle 260 when the needle assembly 242 is nested together. The inflation needle 270 is configured to slide along the longitudinal length 245 of the needle assembly 242 independently from each of the outer needle 250 and the inner needle 260. In at least some embodiments, the inflation-needle 270 defines a stylet lumen 276 suitable for receiving a stylet 280.

When the needle assembly 242 is in the closed position, the inflation needle 270 is retained by the lead lumen (see e.g., 369 in FIG. 3D). In at least some embodiments, the needle assembly 242 is suitable for retaining the inflation needle 270 when the needle assembly 242 is in the open position. As described below, when the needle assembly 242 is transitioned to the open position, an inserted lead passes through the separation channel 268. In at least some embodiments, when the needle assembly 242 is transitioned to the open position, the inflation needle 270 does not pass through the separation channel 268.

In at least some embodiments, an inserted lead has a smaller diameter than the inflation needle 270, with the separation channel 268 having a width sufficient to enable the lead to pass through the separation channel 268, but not the inflation needle 270. In at least some embodiments, the separation channel 268 has a width that is slightly less than the respective diameters of each of the lead and the inflation needle 270; however, the lead is more compressible than the inflation needle 270, thereby enabling the lead to compress enough to pass through the separation channel 268 while preventing the less compressible inflation needle 270 to pass through the separation channel 268.

Optionally, the lead introducer 240 includes a stylet 280 suitable for inserting into the stylet lumen 276 of the inflation needle 270. The stylet 280 has a stylet body 282 attached to a stylet hub 284 along a proximal portion 244 of the needle assembly 242. In at least some embodiments, the stylet body 282 is formed as a rigid wire. The stylet 280 is insertable into the stylet lumen 276 of the inflation needle 270 and configured to slide along the longitudinal length 245 of the needle assembly 242 independently from the needle assembly 242. As described below, the stylet hub 284 may form a portion of the hub assembly 241.

The stylet 280 plugs the stylet lumen 276 to reduce, or even prevent, coring of patient tissue during advancement of the needle assembly 242 into patient tissue. The stylet 280 may be removed from the inflation needle 270 during performance of a placement technique (e.g., a loss of resistance technique). In some instances, a placement technique may not be performed. In which case, the stylet 280 and the stylet lumen 276 may not be necessary. Consequently, in at least some embodiments, the inflation needle 270 does not define a stylet lumen. In which case, the lead introducer 240 may not include the stylet 280.

The needle assembly 242 can be any suitable length for inserting the needle assembly 242 into an epidural space including, for example, 4 inches (approximately 10 cm), 5 inches (approximately 13 cm), 6 inches (approximately 15 cm) as measured from a distal tip of the needle assembly 242 to a distal end of a distal-most hub of the hub assembly 241. The needle assembly 242 can be any suitable gauge including, for example, 14-gauge or 13-gauge.

The outer needle 250, inner needle 260, and inflation needle 270 can be formed from any metal or alloy suitable for inserting into a patient and piercing patient tissue. In at least some embodiments, at least one of the outer needle 250, inner needle 260, or inflation needle 270 is formed from hypodermic tubing. The open channels 256, 266 of the outer needle 250 and inner needle 260, respectively, can be formed from any suitable fabrication procedure. In at least some embodiments, at least one of the open channels 256, 266 is formed by laser cutting.

The hubs 254, 264, 274, 284 can be attached to their corresponding needles/wire 250, 260, 270, 280, respectively, by any suitable technique including, for example, overmolding. The hubs 254, 264, 274, 284 can be fitted together, or mated, within the hub assembly 241. In at least some embodiments, the hubs 254, 264, 274, 284 fit together by resistance fits between adjacent hubs. In at least some embodiments, the hub assembly 241 includes one or more locking mechanisms to facilitate movement of one of the hubs 254, 264, 274, 284 relative to the others.

The inner-needle hub 264 and outer-needle hub 254 are loosely fit together so they may rotate against each other easily. In at least some embodiments, the inner-needle hub 264 and outer-needle hub 254 are designed so a user does not separate the two completely from each other. According, in at least some embodiments, the inner-needle hub 264 is nested completely within the outer-needle hub 254.

In at least some embodiments, the hub assembly 241 includes a luer fitting suitable for receiving a syringe for facilitating a determination of the placement of the needle assembly 242. For example, a luer fitting may enable performance of a loss of resistance technique for determining when the distal portion 243 of the needle assembly 242 enters the patient's epidural space. In at least some embodiments, a luer fitting (not shown) is disposed on the inflation-needle hub 274. In at least some embodiments, when the stylet 280 is not received by the inflation needle 270, the stylet lumen 276 can be used during a placement technique (e.g., a loss of resistance technique).

Turning to FIGS. 3A-3H, one embodiment of a technique for implanting a lead using the lead introducer 240 is described below. The described technique includes use of the optional stylet. FIGS. 3A-3H each show the needle assembly, stylet, and the implanted lead in transverse cross-section.

In FIG. 3A, the needle assembly 242 is shown nested together in an open configuration. The stylet 280 is disposed in the stylet lumen 276 of the inflation needle 270, and the inflation needle 270 is disposed in the inner needle 260 which, in turn, is disposed in the outer needle 250. The open channels 256, 266 of the inner and outer needles 250, 260, respectively, are rotationally-aligned with one another to form the separation channel 268 extending along the longitudinal length of the needle assembly 242.

FIG. 3B shows the needle assembly 242 nested together in a closed position. The stylet 280 is disposed in the stylet lumen 276 of the inflation needle 270, and the inflation needle 270 is disposed in the inner needle 260 which, in turn, is disposed in the outer needle 250. The open channels 256, 266 of the inner and outer needles 250, 260, respectively, are rotationally-offset from one another to close the separation channel 268 and form a lead lumen 369 extending along the longitudinal length of the needle assembly 242.

The closed position can be any rotational offset of the inner needle 260 relative to the outer needle 250 sufficient to prevent a lead, if present in the lead lumen 369, from being able to laterally separate from the needle assembly 242 (i.e., pass out of the lead lumen 369 through the separation channel 268). Although FIG. 3B shows the inner needle 260 rotationally-offset from the outer needle 250 by approximately 90 degrees, other rotational-offsets are sufficient to prevent an inserted lead from being able to laterally separate from the needle assembly 242 including, for example, any amount no less than 30 degrees and no greater than 330 degrees, any amount no less than 45 degrees and no greater than 315 degrees, any amount no less than 60 degrees and no greater than 300 degrees.

The needle assembly can be inserted into the patient with the needle assembly in either the open position or the closed position. During insertion of the needle assembly into a patient, it is typically advantageous for the needle assembly to include a sharpened cutting surface (see e.g., 894 in FIG. 8) along a distal tip to facilitate piercing of patient tissue and advancement of the needle assembly through the pierced tissue. The sharpened cutting surface is typically slanted with respect to the longitudinal length of the needle assembly. Consequently, the outer needle, inner needle, and inflation needle (and, optionally, the stylet) may need to be in a specific rotational orientation relative to one another during advancement of the needle assembly into the patient to create the slant of the sharpened cutting surface. The rotational orientation of the outer needle, inner needle, and inflation needle (and, optionally, the stylet) needed to form the slant of the sharpened cutting surface can be designed so that the inner needle and the outer needle are in either the open position or the closed position. Consequently, the needle assembly can be inserted into the patient with the inner needle and the outer needle in either the open position or the closed position.

The distal portion of the needle assembly is advanced to a location at, or in proximity to, one or more target stimulation locations. Optionally, a placement technique, such as a loss of resistance technique, may be performed to confirm the location of the distal portion of the needle assembly, such as with the patient's epidural space. The stylet 280 may need to be removed from the needle assembly 242 to perform the placement technique.

Once the distal portion of the needle assembly is at the desired location, the inflation needle is removed from the lead lumen, leaving the outer needle and the inner needle within the patient. FIG. 3C illustrates the inflation needle 270 and stylet 280 removed from the lead lumen 369.

As described above, the needle assembly can be advanced within the patient in either the open or closed position. In instances where the needle assembly is advanced while in the open position, the needle assembly is transitioned into the closed position prior to removing the inflation needle. Once the inflation needle (and the optional stylet) is removed from the lead lumen, a lead is inserted into the patient along the lead lumen. FIG. 3D illustrates the lead 312 inserted into the lead lumen 369.

Once the lead is inserted into the lead lumen, the needle assembly is transitioned to the open position. FIG. 3E illustrates the inner needle 260 rotated relative to the outer needle 250 to rotationally align the inner-needle open channel 266 with the outer-needle open channel 256 along the longitudinal length 245 of the needle assembly 242 to form the separation channel 268.

The lead may not readily separate from the needle assembly through the separation channel without facilitation. In at least some embodiments, separation of the lead from the needle assembly is facilitated by displacing the lead with the inflation needle. The hub assembly 241 is arranged with the inflation-needle hub 274 proximal to a proximal end of the separation channel 268. Consequently, insertion of the inflation needle 270 into the lead lumen from a position proximal to the lead causes the inflation needle to wedge the lead through the separation channel 268 from the proximal portion of the needle assembly moving distally.

FIG. 3F illustrates the inflation needle 270 re-inserted into the inner needle 260 and the lead 312 positioned in proximity to the needle assembly 242 at, or in proximity to, a target stimulation location after passing through the separation channel 268.

Once the inflation needle and the stylet are fully re-inserted into the needle assembly, the needle assembly may be transitioned to the closed position to ensure that the lead has been completely separated from the needle assembly. FIG. 3G illustrates the inflation needle 270 and the stylet 280 disposed in the lead lumen 369 and the lead disposed in proximity to the needle assembly 242.

Once the lead is separated from the needle assembly, either the needle assembly can be removed from the patient or the needle assembly may be used to implant one or more additional leads. When the needle assembly is removed from the patient, in some embodiments the outer needle, inner needle, and inflation needle are removed concurrently. In other embodiments, the inflation needle (and optional stylet) are removed prior to removal of the outer needle and inner needle. FIG. 3H illustrates the needle assembly 242 in the closed position and the inflation needle 270 and the stylet 280 removed from the lead lumen 369. The lead 312 is positioned in proximity to the needle assembly 242.

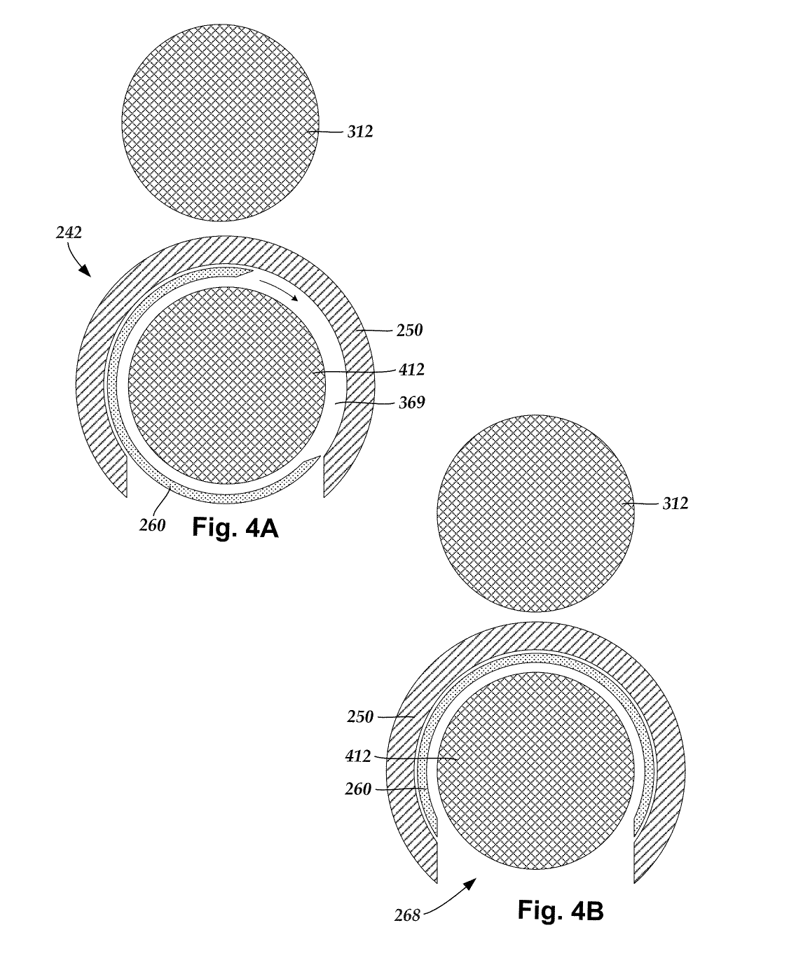

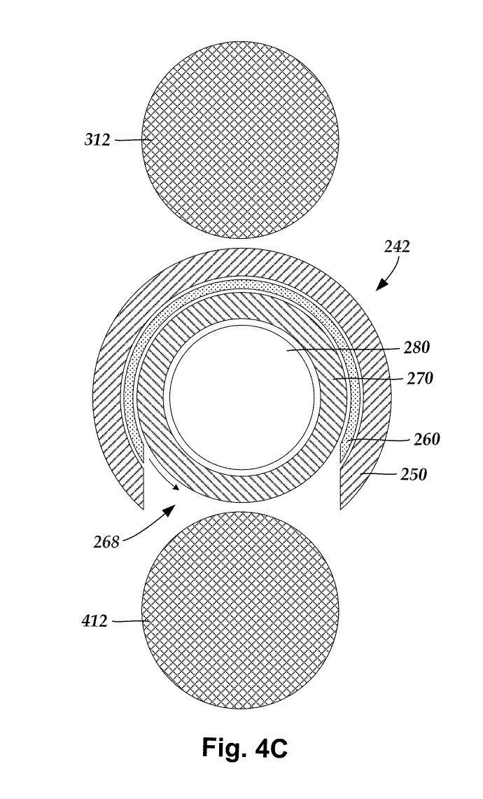

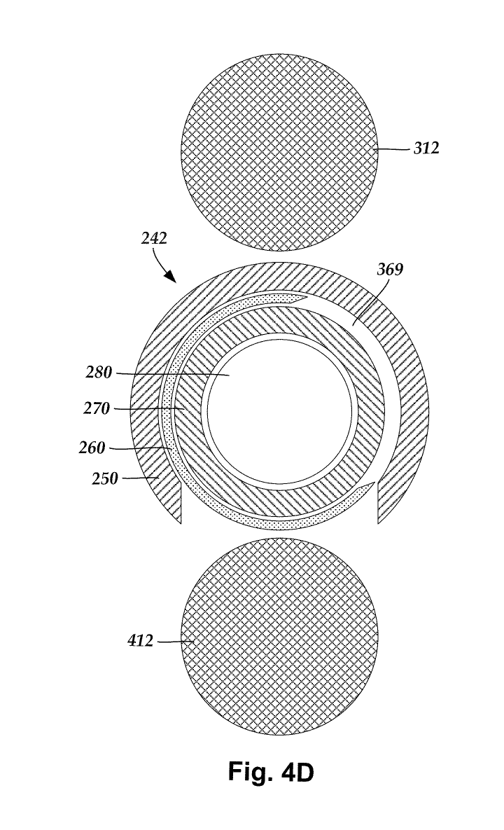

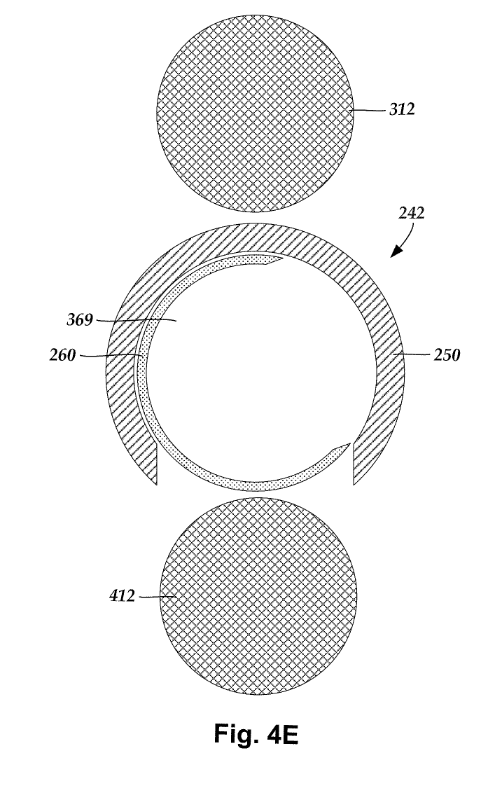

FIGS. 4A-4E show one embodiment of a technique for implanting a second lead into a patient. When the needle assembly is used to implant an additional lead, the additional lead can be implanted without removing the needle assembly from the patient. Additional leads (e.g., a third lead, or more) can subsequently be implanted from the same needle-assembly location, as desired.

FIG. 4A illustrates a second lead 412 inserted into the lead lumen 369. FIG. 4A shows the needle assembly rotated 180 degrees about its longitudinal length from the positioning shown in FIGS. 3A-3H. In some instances, it may be advantageous to rotate the outer needle 250 so that when the lead 412 separates from the needle assembly, the separation will occur at a position that is rotationally-offset from where the previously-implanted lead 312 separated from the needle assembly. FIG. 4A shows the needle assembly rotated 180 degrees from the positioning shown in FIGS. 3A-3H. The needle assembly can be rotated by any suitable amount, depending on the location of a second stimulation location, the amount of space available at the needle-insertion location, or other reasons. In some instances, it may be desirable to not rotate the needle assembly between multiple lead implantations.

Once the second lead is inserted into the lead lumen, the needle assembly is transitioned to the open position. FIG. 4B illustrates the inner needle 260 rotated relative to the outer needle 250 to rotationally align the inner-needle open channel 266 with the outer-needle open channel 256 along the longitudinal length 245 of the needle assembly 242 to form the separation channel 268.

The second lead may not readily separate from the needle assembly through the separation channel without facilitation. In at least some embodiments, separation of the second lead from the needle assembly is facilitated by displacing the second lead with the inflation needle. FIG. 4C illustrates the inflation needle 270 and the stylet 280 re-inserted into the inner needle 260 and the lead 412 positioned in proximity to the needle assembly 242 at, or in proximity to, a second target stimulation location after passing through the separation channel 268 via displacement by the inflation needle 270.

Once the inflation needle and the stylet are fully re-inserted into the needle assembly, the inner needle can be rotated to the closed position to ensure that the second lead has been completely separated from the needle assembly. FIG. 4D illustrates the inflation needle 270 and the stylet 280 disposed in the lead lumen 369 and the lead 412 disposed in proximity to the needle assembly 242.

Once the second lead is separated from the needle assembly, either the needle assembly can be removed from the patient or the needle assembly may be used to implant an additional lead. When the needle assembly is removed from the patient, in some embodiments the outer needle, inner needle, and inflation needle (and optional stylet) are removed concurrently. In other embodiments, the inflation needle, and optional stylet, are removed prior to removal of the outer needle and inner needle. FIG. 4E illustrates the outer needle 250 and inner needle 260 in the closed position and the inflation needle 270 and the stylet 280 removed from the lead lumen 369. The lead 412 is positioned in proximity to the needle assembly 242.

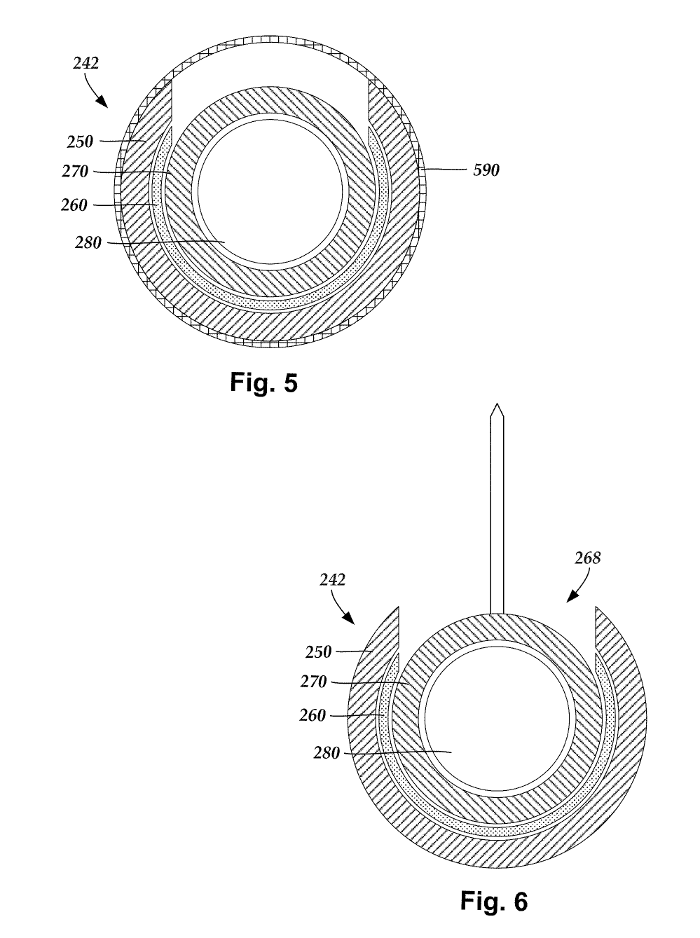

Turning to FIG. 5, in some instances it may be advantageous to dispose a sheath around the needle assembly during lead implantation. The sheath may facilitate performance of a location-finding technique, such as a loss of resistance technique for determining when the distal portion 243 of the needle assembly 242 enters the patient's epidural space. FIG. 5 shows, in transverse cross-section, a sheath 590 disposed around the needle assembly 242. The sheath 590 can be formed from any material suitable for covering the sides of the needle assembly 242 along a portion of the longitudinal length while being inserted into a patient. In some embodiments, the sheath 590 extends along the entire longitudinal length of the needle assembly 242.

The sheath 590 is removed prior to the lead passing through the separation channel during the lead implantation procedure. In at least some embodiments, the sheath 590 is removed by tearing away the sheath 590 from the needle assembly 242. As described below, in at least some embodiments the sheath 590 is cut from the needle assembly 242.

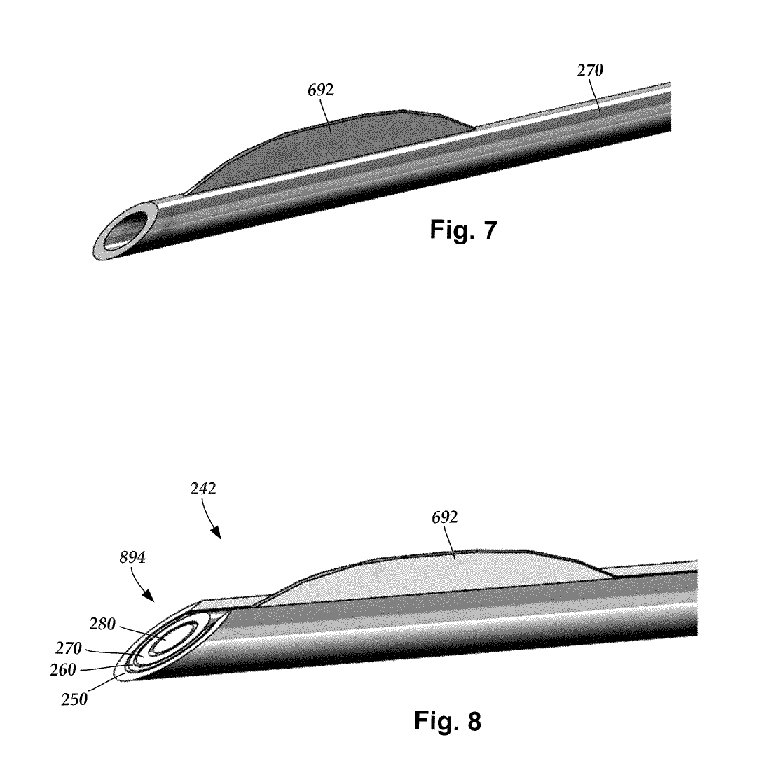

Turning to FIGS. 6-8, in some instances it may be advantageous to create additional space within the patient to implant one or more leads. Additionally, in some instances the sheath may need to be cut away prior to implanting the one or more leads. FIG. 6 illustrates, in transverse cross-sectional view, one embodiment of a blade 692 disposed along a distal portion of the needle assembly 242 while the needle assembly 242 is in the open position, thereby enabling the blade 692 to extend outwardly from the needle assembly 242 through the separation channel 268. FIG. 7 illustrates, in perspective view, one embodiment of the blade 692 disposed along the inflation needle 270. FIG. 8 illustrates, in perspective view, one embodiment of the blade 692 disposed along inflation needle 270 while disposed in the inner needle 260.

In FIGS. 6-8, the blade 692 is shown disposed on the inflation needle 270. In alternate embodiments, the blade 692 can be disposed along a cutting member that is removably insertable into the inner needle 260 of the needle assembly during a lead implantation procedure. In at least some embodiments, the cutting member is similar in structure to the stylet 280.

The distance that the blade 692 extends outwardly from the needle assembly 242 can be any suitable length for cutting the sheath 590 or for cutting tissue in proximity to the needle assembly 242 to create space for implanting one or more leads.

FIG. 8 additionally shows a sharpened cutting surface 894 disposed along the distal portion of the needle assembly 242. The sharpened cutting surface 894 is used for piercing patient tissue to facilitate advancement of the needle assembly into a patient. The sharpened cutting surface 894 is typically formed at a slant with respect to the longitudinal length of the needle assembly 242 to facilitate piercing of patient tissue. In at least some embodiments, the blade 692 is aligned with the slant of the sharpened cutting surface 894 to ensure that when the outer needle 250, inner needle 260, and inflation needle 270 are rotationally aligned to form the slanted, sharpened cutting surface 894, the needle assembly 242 is in the open position and the blade 692 extends through the separation channel 268.

Optionally, the needle assembly may include a bend, or bezel, (not shown) disposed along the distal portion of the needle assembly proximal to the sharpened cutting surface 894. The bend may be useful for facilitating maneuvering of the distal portion of the needle assembly within patient tissue.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A lead introducer comprising
a needle assembly having a distal portion, a proximal portion, and a longitudinal length, the needle assembly comprising
an outer needle defining an outer-needle open channel extending longitudinally along the needle assembly, and
an inner needle defining an inner-needle open channel extending longitudinally along the needle assembly, the inner needle disposed within the outer needle and rotatable independently within the outer needle to transition the needle assembly between an open position and a closed position, wherein when the needle assembly is in the open position the inner-needle open channel is rotationally-aligned with the outer-needle open channel to form a separation channel for separating a lead disposed in the needle assembly from the needle assembly, wherein when the needle assembly is in the closed position the inner-needle open channel is rotationally-offset from the outer-needle open channel and the outer needle and inner needle define a lead lumen within the needle assembly; and
a hub assembly coupled to the proximal portion of the needle assembly, the hub assembly comprising
an outer-needle hub coupled to the outer needle, and
an inner-needle hub coupled to the inner needle.

2. The lead introducer of claim 1, further comprising an inflation needle configured and arranged to slide within the inner needle.

3. The lead introducer of claim 2, further comprising a stylet configured and arranged to slide within the inflation needle.

4. The lead introducer of claim 2, wherein the distal portion of the needle assembly comprises a sharpened cutting surface configured and arranged to facilitate piercing of patient tissue.

5. The lead introducer of claim 2, further comprising a blade extending outwardly from the needle assembly, the blade configured and arranged to slide longitudinally along the needle assembly and cut away patient tissue in proximity to the needle assembly.

6. The lead introducer of claim 5, wherein the blade is attached to the inflation needle.

7. The lead introducer of claim 5, wherein the blade is attached to a cutting member configured and arranged to slide within the inner needle.

8. The lead introducer of claim 1, further comprising a sheath configured and arranged for disposing over the needle assembly.

9. The lead introducer of claim 1, wherein the hub assembly comprises a rotation actuator for facilitating manual transitioning of the needle assembly between the open position and the closed position by a user of the lead introducer.

10. The lead introducer of claim 9, wherein the hub assembly comprises a rotation-control feature coupled to the rotation actuator, the rotation-control feature configured and arranged for facilitating transitioning of the needle assembly between the open position and the closed position.

11. The lead introducer of claim 10, wherein the rotation-control feature is configured and arranged to releasably lock the needle assembly in at least one of the open position or the closed position.

12. The lead introducer of claim 10, wherein the rotation-control feature comprises a biasing member configured and arranged for biasing transitioning of the needle assembly between the open position and the closed position.

13. The lead introducer of claim 1, wherein the lead introducer is configured and arranged for passing a lead disposed in the needle assembly through the separation channel by displacing the lead with the inflation needle.

14. An insertion kit comprising:
the lead introducer of claim 1; and
an electrical stimulation lead comprising a plurality of electrodes disposed along a distal portion of the electrical stimulation lead.

15. An electrical stimulation system comprising:
the insertion kit of claim 14; and
a control module coupleable to the electrical stimulation lead, the control module comprising
a housing, and
an electronic subassembly disposed in the housing.

16. A method for implanting an electrical stimulation lead into a patient, the method comprising:
(a) advancing the lead introducer of claim 1 into the patient;
(b) removing the inflation needle from the patient, leaving the inner needle and outer needle within the patient, the inner needle and outer needle being in the closed position, thereby forming the lead lumen extending along the longitudinal length of the needle body;
(c) inserting into the lead lumen a first electrical stimulation lead comprising a plurality of electrodes disposed along a distal portion of the first electrical stimulation lead and a plurality of terminals disposed along a proximal portion of the first electrical stimulation lead;
(d) rotating the inner needle relative to the outer needle to transition the needle assembly to the open position, thereby forming the separation channel extending along the longitudinal length of the needle body; and
(e) removing the needle assembly from the patient, leaving the first electrical stimulation lead within the patient.

17. The method of claim 16, further comprising cutting away patient tissue in proximity to the needle assembly using a blade extending outwardly from within the needle assembly.

18. The method of claim 16, wherein (d) rotating the inner needle relative to the outer needle to transition the needle assembly to the open position comprises using a rotation actuator.

19. The method of claim 16, further comprising reinserting, subsequent to performing step (d) and prior to performing step (e), the inflation needle into the lead lumen, the reinsertion of the inflation needle into the lead lumen displacing the first electrical stimulation lead from the needle assembly, thereby causing the first electrical stimulation lead to separate from the needle assembly through the separation channel.

20. The method of claim 16, further comprising:
transitioning, subsequent to performing step (d) and prior to performing step (e), the needle assembly to the closed position;
removing the inflation needle from the needle assembly;
inserting into the lead lumen a second electrical stimulation lead comprising a plurality of electrodes disposed along a distal portion of the second electrical stimulation lead and a plurality of terminals disposed along a proximal portion of the second electrical stimulation lead; and
transitioning the needle assembly to the open position.
